(19) United States
(12) Patent Application Publication
(10) Pub. No.: US 2025/0201337 A1
(43) Pub. Date: Jun. 19, 2025

(54) SYSTEMS AND METHODS USING NEURAL NETWORK DISCRIMINATIVE FEATURE LOCALIZATION TO DETERMINE PROTEIN AND LIGAND FUNCTIONAL STRUCTURE

(71) Applicant: Stephen Gbejule Odaibo, Sugar Land, TX (US)

(72) Inventor: Stephen Gbejule Odaibo, Sugar Land, TX (US)

(73) Assignee: Deep EigenMatics LLC, Sugar Land, TX (US)

(21) Appl. No.: 19/067,865

(22) Filed: Mar. 1, 2025

(51) Int. Cl. G16B 15/30; G06N 3/09

(52) U.S. Cl. CPC G16B 15/30; G06N 3/09

(57) ABSTRACT

Methods, systems, and apparatus for determining a conformational structure of a protein by using discriminative feature localization to iteratively update the protein structure locally, optimizing with respect to a physical or biological property of the structure representation. In one aspect, a method comprises initialization a plurality of structure parameters, selecting a physical or biological property of interest, training a neural network to score protein structural conformations on their measure of the selected property, using the neural network to perform inference yielding both a classification score and a discriminative feature localization map; and iteratively updating the structure parameters over the discriminative feature map, optimizing with respect to the physical or biological property of interest.

### FIELD OF THE INVENTION

The present invention relates generally to ML (Machine Learning)/AI (Artificial Intelligence) determination of protein and ligand structure; and particularly to use of discriminative feature localization mapping to determine functionally relevant protein and ligand structure conformations.

### BACKGROUND OF THE INVENTION

Proteins consist of chains of amino acids. The biological functionality and effects of a protein depends exquisitely on the conformational structure it assumes upon folding. Furthermore, protein structures are protean; not having a unique conformation, but instead changing depending on their initial conditions as well as their local microenvironmental milieu.

The various structural conformations (or “states”) a given protein can assume have different functional roles and different levels of biological significance. As such, for purposes such as drug discovery and development of targeted genetic therapies for instance, certain conformations of a protein may be of greater relative interest than other conformations.

Notably, however, proteins despite their protean nature and multiple possible structural conformations, are also physically constrained within certain bounds. For instance, a G-protein-coupled receptor (GPCR) is a membrane-bound receptor that crosses the cell-membrane 7 times, hence its other moniker “7-transmembrane” receptor. Certain of its loops are intracellular, while others are extracellular. In many instances, changes to GPCR proteins are constrained within the 7-transmembrane bounds. More generally, the differences between biologically interesting structural conformations of a protein are typically constrained in structural parameter space.

Therefore, given one biologically relevant stable predicted structural conformation of a protein whereby that stable predicted conformation lacks a certain known biological and or physical property; it is of great importance to be able to determine a different stable predicted conformation that has a greater measure of the property of interest. The different stable conformation, however, must also respect the physical and biological constraints of the protein and its environment. This preserves the biological relevance of the predicted structural conformation and increases the likelihood of its usefulness for drug discovery and targeted therapy development.

Various machine learning methods have been applied towards problems in protein folding and protein design. However, the biological nuance of respecting tertiary, quaternary, and microenvironmental constraints while determining protein structure—and particularly different structural conformations of a given protein—has generally not been properly recognized or addressed. It however is critical to determining protein structural conformations that will most likely yield novel and effective drugs and therapies.

Since proteins are much more than a sequence of amino acids existing in isolation. To decipher their functional structure using in-silico methods such as machine learning techniques, those techniques must somehow remain cognizant of the complexity of the in vivo environment in which proteins exist and function.

Here, we disclose systems, methods, and apparatus by which to determine biologically-relevant protein structure conformations. Given a protein structure representation lacking a certain property of interest, we will say that protein structure is of the “inverse state” (with respect to that property), while we will say the corresponding structures having the property of interest are of the “State of interest.” Here, we disclose a means of determining a structure representation of the state of interest of a protein by locally updating only the structural parameters that need to be changed to confer that protein with the property of interest. Starting with a structure in the inverse state, we determine the discriminating structural features that make the given structure be in the inverse state, and we iteratively update those features alone (i.e. “locally”), thereby changing the resultant structure towards the state-of-interest.

We utilize neural network discriminative feature localization to determine the discriminating features that distinguish between the inverse state and the state-of-interest, and then we use a localization update optimization method to iteratively change those sets of features towards the state of interest.

Discriminative feature localization methods are methods for localizing the region within data that causes a machine learning classifier to classify that data instance as belonging to one class vs another. By way of example and not limitation, discriminative feature localization methods include Class Activation Mapping (CAM), CAM-variants, and occlusion sensitivity analysis.

The designation of state-of-interest vs the inverse state depends entirely on the underlying objectives and biology. Consider for instance, a given receptor with primarily two equilibrium structural conformations (e.g. bound state vs unbound state), then if the bound state therapeutically increases downstream signaling substrates, the bound state structural conformation may be designated as being of the state-of-interest. On the other hand, the unbound structural conformation would be designated as being of the inverse state.

There have been many instances of deep learning methods applied to general problems in this field. In particular, (i) the problem of protein folding, i.e. determining the structure of a given sequence of amino acids, and (ii) the problem of protein design, i.e. predicting the amino acid sequence that satisfies a given desired property. However, none of these instances addressed the particular problem of conserving structure outside of the class discriminating aspect, and none utilized the systems, methods, and apparatus as disclosed herein.

Of particular note, there have been prior instances of class activation mapping applied towards identifying what specific amino acids or groups of amino acids in a protein are critical for certain biological functions of that protein. This approach is generally a form of biological activity analysis as a function of constituent amino acids—the general objective there being to explain and annotate protein function. See for instance Gligorijević, Vladimir, et al. “Structure-based protein function prediction using graph convolutional networks.” Nature communications 12.1 (2021): 3168. Or Pu, Limeng, et al. “DeepDrug3D: Classification of ligand-binding pockets in proteins with a convolutional neural network.” PLOS computational biology 15.2 (2019): e1006718.

However, prior to this invention disclosure, there have been no instances where class activation mapping or other discriminative feature mapping methods have been used to determine protein structure, and particularly to determine the predicted conformational structure as disclosed herein.

Specifically, prior to the disclosure of this invention, there were no machine learning methods for protein structure determination using discriminative feature localization to identify the subset of structural parameters to iteratively update, and thereby optimize towards a physical or biological property.

### OBJECTS OF THE INVENTION

It is an object of this invention to provide a system, method, and apparatus for protein and ligand functional structure determination, whereby the system uses discriminative feature localization mapping to identify what subset of structural parameters to iteratively update, and thereby optimize towards a selected physical or biological property.

Yet other objects, advantages, and applications of the invention will be apparent from the specifications and drawings included herein.

### SUMMARY OF THE INVENTION

Given the representation of a protein structure whereby the protein structure representation lacks a certain property of interest, the invention disclosed herein provides a system to determine a corresponding conformational structure for that protein such that the determined structure has a greater measure of the property of interest.

The structure of the protein can be represented via a set of structure parameters. For instance, the spatial [x,y,z] coordinates of representative atoms in each amino acid backbone may be chosen. By similar token, a distance map may be used to represent the protein structure; such that the distance map is itself represented as a matrix (D) of size N² where N is the number of amino acids constituting the protein. The (i, j)th entry of the D matrix represents the distance between representative atoms of the ith and jth amino acids. Another parametric representation is via torsion angles (ϕ, φ) between the amino acids of the protein. Furthermore, the structure representation can be probabilistic, whereby for instance the distance between any two amino acids is represented as a gaussian centered about the mean and of a specified variance. Similarly for torsion angle representation, whereby the angles may be taken as the means of a gaussian for instance. Any other appropriate parametric representation adequately capturing the structure of the protein can be utilized.

The assessment of a given protein structure representation's measure of the property of interest is done using a neural network. Furthermore, the neural network is equipped with a class discriminative feature localization mechanism, with which it identifies the aspects of the input protein structure representation that caused the classification into one class (e.g. inverse state) vs the other (e.g. state of interest). We will call this neural network, a State Determining Discriminative Classifier (SDDC).

The SDDC neural network may be a graphical neural network, a graphical convolutional neural network, a convolutional neural network, a recurrent neural network, a transformer-based network architecture, or any other neural network configuration or architecture that enables representation of proteins in a space where meaningful physical and biological classification can be conducted.

Training of the SDDC neural network relies on a protein structure database which includes multi-dimensional indexing across associated properties. In particular, each of the possible values of each property's random variable should be represented in a statistically representative manner in the database. For instance, consider a simple example of a protein with primarily two stable structural conformations at equilibrium (e.g. a ‘bound state’ and an ‘unbound state’). The protein structure database for training the SDDC should contain a plurality of diverse protein structures in the bound state as well as a plurality of diverse protein structures in the unbound state. Furthermore, the database should be sufficiently large enough and sufficiently diverse enough to encode a learnable pattern which the SDDC will learn. After training, given as input a protein structure representation previously unseen to the SDDC, it outputs its classification prediction (i.e. bound state vs unbound state). Of note, the SDDC need not be binary—it can be multiclass—and it need not be a scalar output, it can be a vectorized form, a molecule, protein, or any architectural output appropriately representing the problem being studied.

In addition to the SDDC yielding the property classification of the protein conformation as output, it also yields the discriminative feature localization map, a ‘mask,’ effectively subsegmenting the aspects of the structure parameters that determined the property class. Without loss of generality, assume by way of example and not limitation, that the output class is the inverse state. This localization of the discriminative features will serve as the target for local updates in the next step for the purpose of iteratively moving the property class from the inverse state towards the state of interest.

The discriminative feature localization mechanism can be any method that enables localization of the particular features in the input protein structure representation that decided the property class. By way of example and not limitation, discriminative feature localization methods include Class Activation Mapping (CAM) and CAM-variants. As used in this description and in the appended claims, the term CAM-variant means any method that uses a decomposition of the neural network's feature extraction, weighted scalings, and activations to determine the discriminative feature map. Examples of CAM-variants include but are by no means limited to Gradient-weighted Class Activation Mapping (Grad-CAM), Guided Grad-CAM, Guided Backpropagation, Integrated Gradients, Eigen-CAM, Self-Matching CAM, Grad-CAM++, Smooth Grad-CAM++, Score CAM, Ablation-CAM, Layer-wise Relevance Propagation (LRP), and Shap-CAM.

Another type of method of discriminative feature localization is occlusion sensitivity analysis.

In one embodiment of the invention, the discriminative feature localization method is a Class Activation Map (CAM). These may use a Global Average Pooling (GAP) step following a series of feature extraction steps. In particular, given a protein structure representation as input, the SDDC layers serve as feature extractors yielding a set of feature maps. Each feature map can be condensed into a single scalar via a global average pooling operation, for instance. Together, the set of feature maps therefore becomes a feature vector after the GAP operation. The feature vector may be connected via a densely connected (“Fc7”) layer to an output node activated by a Rectified Linear Unit (ReLU) or similar activation function. Since the ReLU family of activations are monotonically increasing over positive input domain and zero otherwise, it follows that classification into a given class occurs when scaled inputs from the feature vector are positive. This in turn occurs when the scaled feature maps are positive and higher than those of the non-selected class. The scaled feature maps can be upsampled and overlaid on the input protein representation to identify the aspects of the structural parameters that determined the classification.

Upon identifying the discriminative features, the next step is to pass the protein structure and associated discriminative feature map as input into a Localized Structure Update Engine. This yields an updated protein structure whereby only the discriminative feature maps are changed from the input structure. Furthermore, at convergence, the updated structure is of the state of interest and no longer of the inverse state.

The Localized Structure Update Engine consists of the SDDC neural network as well as a localized structure update method. The localized structure update method could be any number of methods including but not limited to genetic algorithms and variants, particle swarm optimization methods and variants, simulated annealing and variants, and stochastic gradient descent and variants. As noted, in some embodiments it could be a genetic algorithm whereby the SDDC evaluates and checkpoints the property value following a certain number of iterations. A similar checkpointing forward-facing approach can be applied to particle swarms with the trained SDDC as value function. Additionally, as noted, stochastic gradient descent (SGD) may also be utilized. SGD is a known and effective method for updating structural parameters based on a gradient of those parameters derived from a neural network (Ingraham, John, et al. “Learning protein structure with a differentiable simulator.” International Conference on Learning Representations. 2018.)

In summary, the invention disclosed herein consists of systems, methods, and apparatus to use discriminative feature localization to determine protein structure. In particular, a State Determining Discriminative Classifier (SDDC) neural network is trained on a multidimensionally indexed protein structure database. The SDDC is equipped with a discriminative feature localization mechanism. The trained SDDC acts on a protein structure representation to yield its property classification as well as discriminative feature map. A Localized Structure Update Engine is used to update the discriminative features alone in a manner that yields a corresponding structure optimized towards the opposite class. Yet by making no changes outside the discriminative feature maps, the update procedure preserves respect for the biological and physical constraints on the input protein structure. Upon determining the desired protein structural conformation optimized towards the state of interest, the conformation can be applied to a variety of objectives including but not limited to pharmacological drug discovery, candidate ligand search, and protein design.

The invention consists of several outlined processes below, and their relation to each other, as well as all modifications which leave the spirit of the invention invariant. The scope of the invention is outlined in the claims section.

### BRIEF DESCRIPTION OF THE DRAWINGS

In the following detailed description of the invention, we reference the herein listed drawings and their associated descriptions, in which:

FIG. 1 is an example of a protein structure representation;

FIG. 2 is an example of an SDDC neural network;

FIG. 3 is an example of an SDDC neural network;

FIG. 4 is an example of an SDDC with Class Activation Map (CAM);

FIG. 5 is an illustration of CAM showing discriminative feature overlay;

FIG. 6 is an example of a localized structure update yielding an unbound to bound state transition;

FIG. 7 is an example of a localized structure update yielding more generally a ‘state of interest’;

FIG. 8 is a schematic illustration of a localized structure update engine;

FIG. 9 is a schematic illustration of SDDC training and localized structure update; and

FIG. 10 is an example of a computing environment.

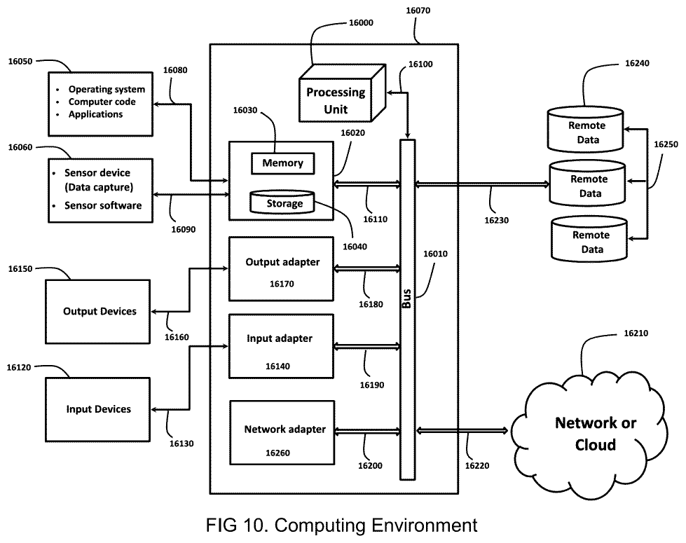

### DETAILED DESCRIPTION OF THE PREFERRED EMBODIMENTS

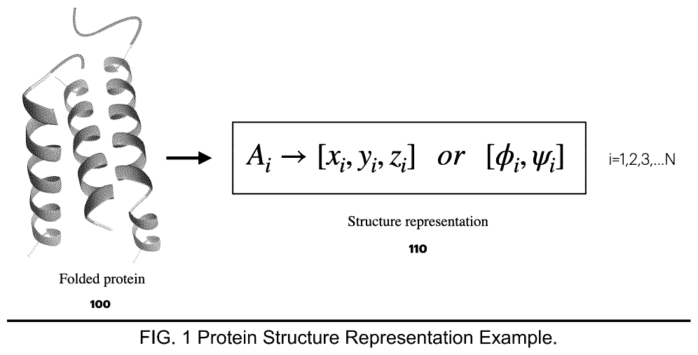

The illustration in FIG. 1 is a preferred embodiment of a protein structure representation. The folded protein 100 can be represented as shown 110 such that for each amino acid, the spatial [x,y,z] coordinates of representative atoms in each amino acid backbone may be chosen. Alternatively, a pairwise distance map may be used to represent the protein structure; such that the distance map is itself represented as a matrix (D) of size N² where N is the number of amino acids constituting the protein; wherein the (i, j)th entry of the D matrix represents the distance between representative atoms of the ith and jth amino acids.

Another method of representing the protein structure is via torsion angles, (¢, q), between the amino acids of the protein.

Furthermore, the structure representations can be probabilistic, whereby for instance the distance between any two amino acids is represented as a gaussian centered about the mean and of a specified variance. Similarly for torsion angle representation, whereby the angles are taken as the means of a gaussian, for instance. Any other appropriate representation adequately capturing the structure of the protein can be utilized.

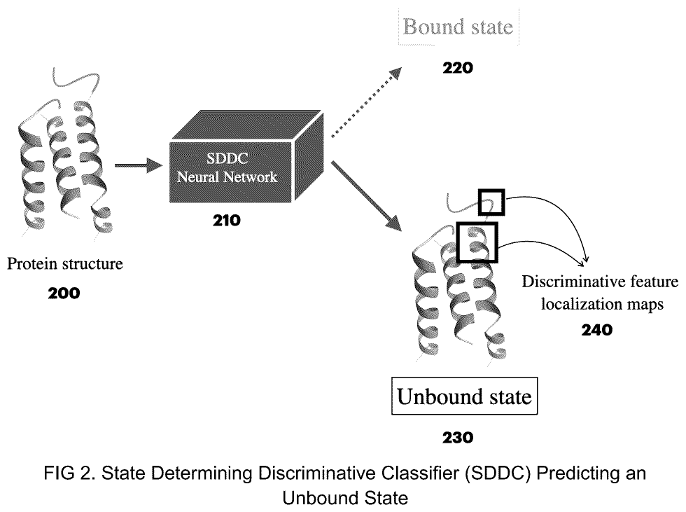

The exemplary illustration in FIG. 2 depicts a protein structure representation 200 passed as input into an SDDC neural network 210 which classifies the input protein structure as being of the unbound state 230. Furthermore, the SDDC localizes the discriminative feature maps 240. The bound state is also shown 220. The SDDC neural network 210 may be a graphical neural network, a graphical convolutional neural network, a convolutional neural network, a recurrent neural network, a transformer-based network architecture, or any other neural network configuration or architecture that enables representation of proteins in a space where meaningful physical and biological classification can be conducted.

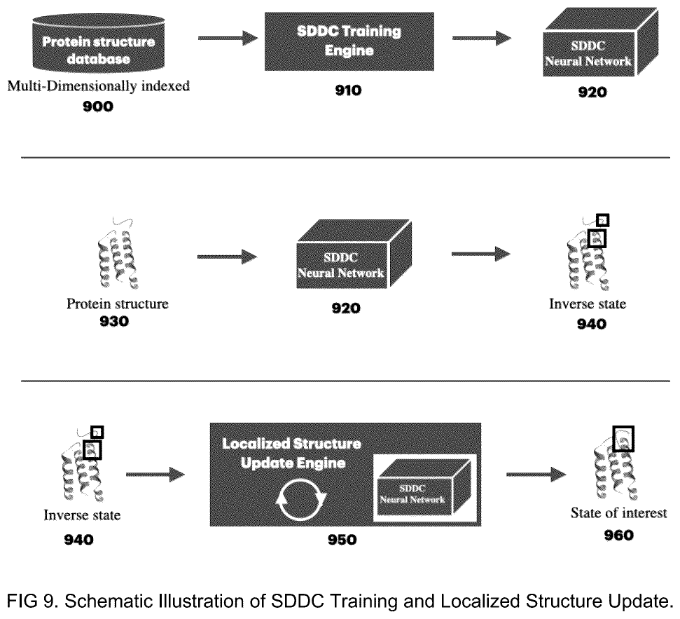

Training of the SDDC neural network 210 relies on a protein structure database 900 depicted in FIG. 9. The database includes multi-dimensional indexing across associated properties. In particular, each of the possible values of each property's random variable should be represented in a statistically representative manner in the database. For instance, as depicted in FIG. 2, consider a simple example of a protein with primarily two stable structural conformations at equilibrium (e.g. a ‘bound state’ and an ‘unbound state’). The protein structure database 900 for training the SDDC should contain a plurality of diverse protein structures in their respective bound states as well as a plurality of diverse protein structures in their respective unbound states. Furthermore, the database 900 should be sufficiently large and sufficiently diverse to encode a learnable pattern which the SDDC 210 will learn. After training, given as input a protein structure representation previously unseen to the SDDC, it outputs its classification prediction (i.e. bound state vs unbound state). In the example depicted in FIG. 2., the SDDC is a binary classifier. However, in other embodiments, the SDDC may be multiclass.

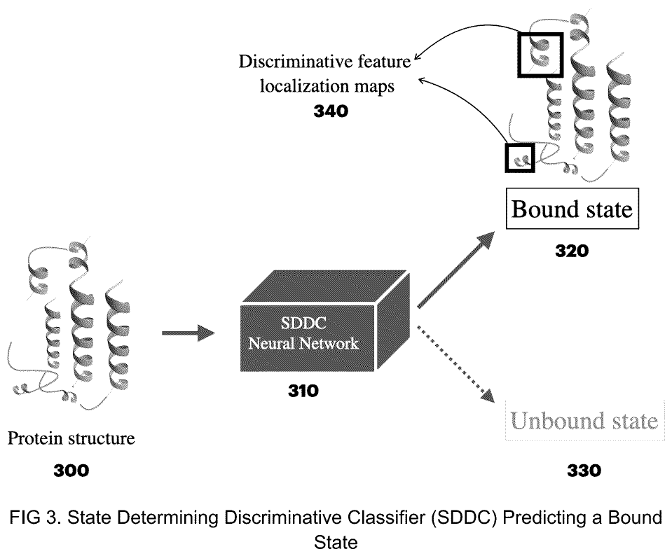

The exemplary illustration in FIG. 3 depicts a protein structure representation 300 passed as input into an SDDC neural network 310 which classifies the input protein structure as being of the bound state 320. The discriminative feature maps 340 accompany the classification output.

FIG. 2 and FIG. 3 both depict an example whereby the SDDC neural network is a binary classifier that has been trained such that given a protein structure representation, it infers whether that input structure is of the bound vs the unbound state. Of note, the SDDC may be trained instead to infer other properties from input protein structure. The property (or properties) which the SDDC is trained to evaluate depend on the objective. The training database simply needs to be representative of the property values, and must contain a sufficiently large and sufficiently representative diverse plurality of structural conformations across property values.

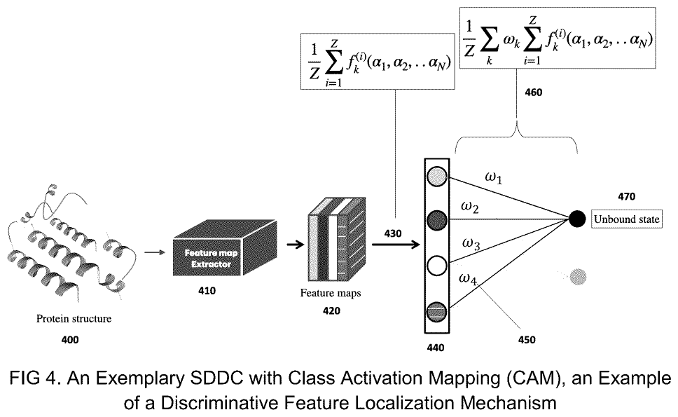

FIG. 4 depicts a preferred embodiment of a discriminative feature localization method of an SDDC neural network. In this example, the discriminative feature localization method is a Class Activation Mapping (CAM) and the SDDC has a Global Average Pooling (GAP) layer 430 for CAM. The feature extraction layers 410 act on an input protein structure representation 400 to yield a set of feature maps 420. For each feature map, its values fₖ(α₁, . . . , α_{N}) are globally averaged as shown in 430 to yield a single entry in the feature vector 440. Where fₖ(α₁, . . . , α_{N}) is the kth feature map, αₚ is the pth axis variable of the feature map's domain wherein the pth axis is of dimension dim(αₚ). The total number of elements in each feature map is denoted Z and is given by,

$$Z = {\prod }_{p = 1}^{N} dim ( {α}_{p} )$$

The feature vector 440 has as many entries as there are feature maps in the preceding layer 420. The feature vector is connected via a dense layer to the output scores as shown in 460. An activation function such as ReLU may be applied to the output scores, the higher of which is the output classification as illustrated in 470. It follows that since ReLU is monotonically increasing over the positive domain, the higher of the two outputs necessarily has the higher raw score computed via the formula in 460. One may factor in the weights ωₖ as follows:

$${\sum }_{k} {ω}_{k} ( \frac{1}{Z} {\sum }_{i = 1}^{Z} {f}_{k}^{( i )} ( {α}_{1} , … , {α}_{N} ) ) = \frac{1}{Z} {\sum }_{k} {\sum }_{i = 1}^{Z} ( {ω}_{k} {f}_{k}^{( i )} ( {α}_{1} , … , {α}_{N} ) )$$

whereby ωₖfₓ(α₁, . . . , α_{N}) are weighted feature maps for which given a ReLU-like activation only the non-negative values contribute towards the classification and they do so proportionally, i.e. the higher the value of a point in the weighted feature map, the more it contributes towards the final output score of its associated class. This property is preserved through the global average pooling of the weighted feature maps. Summing over the k weights associated with a given class is equivalent to a discriminative map overlay.

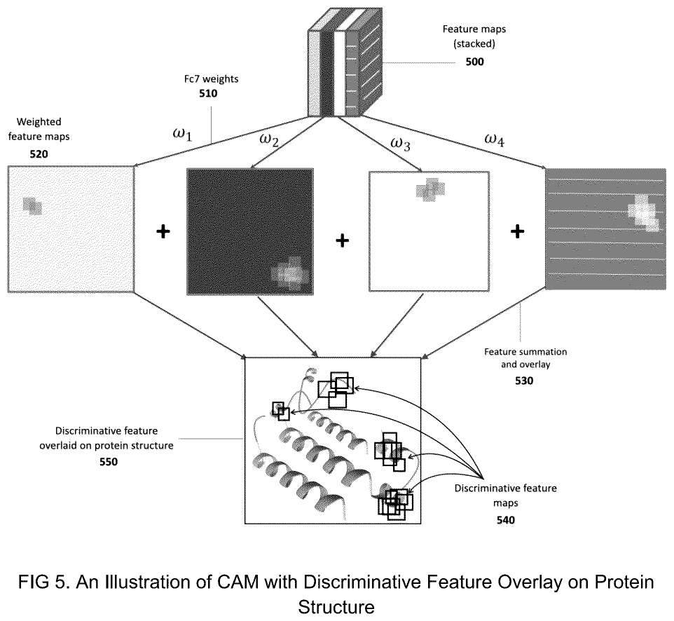

FIG. 5 elucidates a class activation mapping mechanism embodiment of discriminative feature localization. The final layer weights—i.e. the weights by which the feature maps are scaled—are shown in 510, yielding the weighted feature maps 520. The weighted feature maps are then upsampled back to the size of the original input protein structure representation data 530 and are all overlaid on the representation as shown in 550. The discriminative feature maps 540 are directly superimposed on the input data after upsampling.

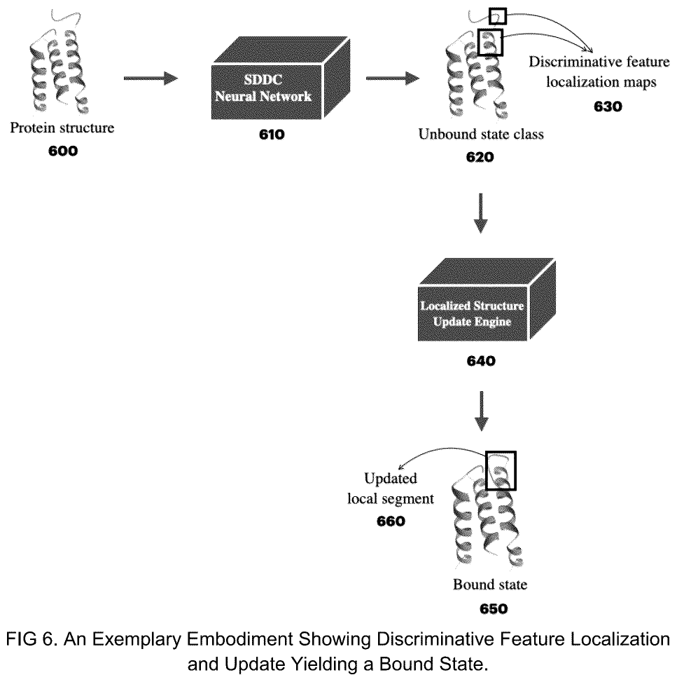

FIG. 6 shows an exemplary schematic of an SDDC neural network 610 trained to distinguish between bound and unbound structural conformations. In the illustration, the SDDC acts on a protein structure representation 600 and yields an output class of ‘Unbound state’ 620 and also outputs the corresponding discriminative feature maps 630. The Localized Structure Update Engine 640 acts on the SDDC output 620 to locally update its structure only where the discriminative mask indicates. The output 650 of the Localized Structure Update Engine at convergence is of the bound state. The updated local segment 660 has a different structural conformation than the corresponding segments of the input structure 620. All other aspects of the output 650 are identical to the input 620.

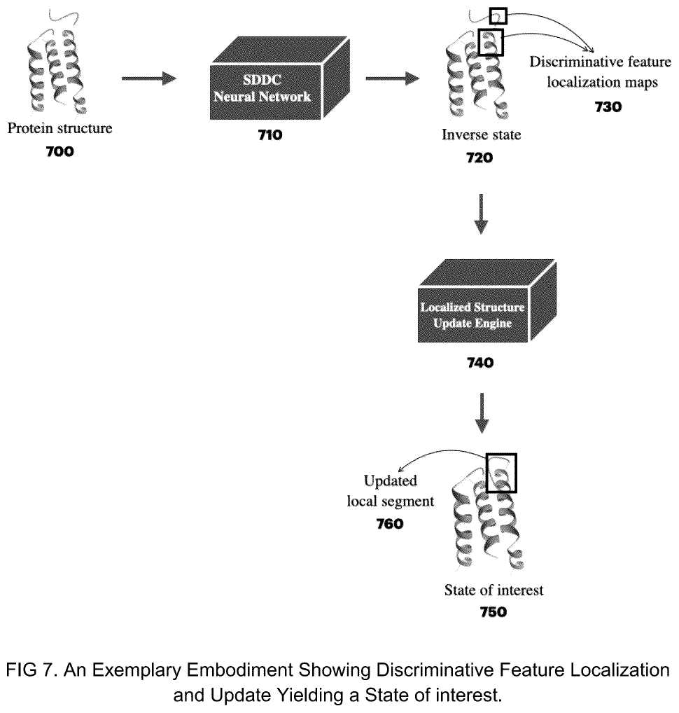

The embodiment illustrated in FIG. 6. involves a simple binary classifier SDDC neural network whose two classes are ‘bound state’ and ‘unbound state’ respectively. Of note, the SDDC in the invention disclosed herein may be a multiclass classifier trained to classify an input protein structure according to any number of a plurality of properties. FIG. 7 illustrates this generalizability by depicting an SDDC 710 which acts on an input protein structure 700 and in this example infers that 700 belongs to the inverse state class. The SDDC-labeled protein conformation 720 along with its associated discriminative feature maps 730 are then passed as input into the Localized Structure Update Engine 740. At convergence, the output 750 from the update engine belongs to the ‘state of interest’ class. It differs from the input 720 only locally, i.e. over the discriminative feature maps.

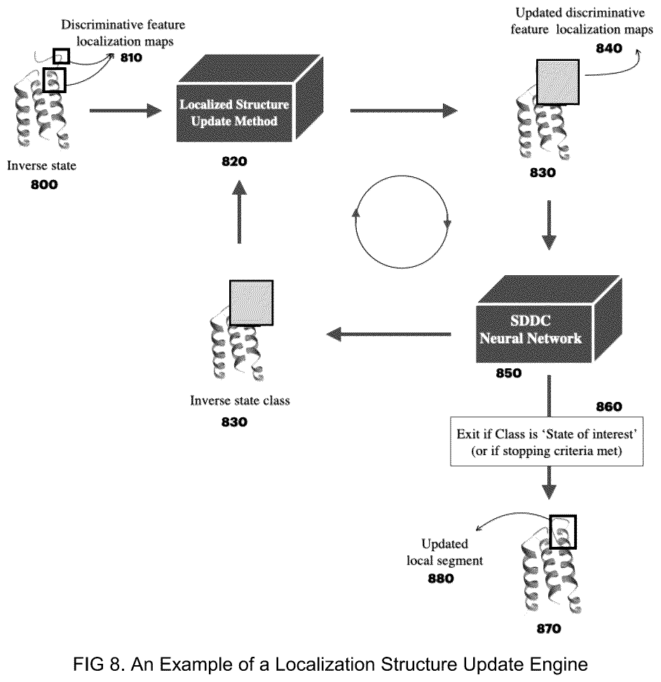

An exemplary embodiment of a Localized Structure Update Engine is depicted in FIG. 8. It involves as components, a localized structure update method 820 and a trained SDDC neural network 850. A protein structure representation SDDC-labeled as belonging to Inverse state class is passed in along with its discriminative feature maps 810 as input into the localized structure method 820. The localized structure method acts on the input to locally update only the aspects indicated by the discriminative feature maps. This results in an updated local segment 840. The updated protein structure 830 is then passed as input into the SDDC 850. If the updated structure 830 is found to be of the ‘state of interest’ as desired, the engine exits and outputs the updated structure representation 870 along with a mask of the updated aspects 880. If however, the resulting class is still of the inverse state and the stopping criteria (e.g. max number of iterations) is not yet met, then the updated structural conformation 830 is passed as input into the localized structure update method 820. The process continues in a loop as described till exit condition 860 is met. When exit condition 860 is met, the engine exits and outputs the most updated structural conformation 870.

The localized structure update method 820 could be implemented in any number of ways including but not limited to genetic algorithms and their variants, particle swarm optimization methods and their variants, simulated annealing methods and their variants, stochastic gradient descent and its variants, and Monte Carlo Tree Search (MCTS) methods and their variants. The key principle is simply to progressively move the SDDC score towards the state of interest and away from the inverse state. The progression of the score change need not be monotonic, either, but simply needs to progress in an expectation sense. For instance, random walk with pull type schema may not progress monotonically, but in aggregate (i.e. in an expectation sense) they progress in the correct direction.

As noted, in some embodiments the Localized Structure Update Method could be a genetic algorithm whereby the SDDC evaluates and checkpoints the property value following a certain number of iterations. A similar checkpointing forward-facing approach can be applied to particle swarms with the trained SDDC as value function. Additionally, as noted, stochastic gradient descent (SGD) may also be utilized. SGD is a well known and effective method for updating structural parameters based on a gradient of those parameters derived from a neural network.

FIG. 9 shows a schematic illustration of the SDDC training, the trained SDDC in action, and the Localized Structure Update Engine function. A multi-dimensionally indexed protein structure database 900 can be used by the SDDC Training Engine 910 to produce a trained SDDC 920. In this exemplary embodiment, the trained SDDC is shown acting on a protein structure representation 930 to yield an output 940 classified as being of the ‘inverse state’. The output 940 includes discriminative feature masks and is passed as input into a Localized Structure Update Engine 950, yielding a final output 960 classified as being in the state of interest.

Ones with ordinary skill in the art will recognize that the invention disclosed herein can be implemented over an arbitrary range of computing configurations. We will refer to any instantiation of these computing configurations as the computing environment. An exemplary illustration of a computing environment is depicted in The Computing Environment FIG. Examples of computing environments include but are not limited to desktop computers, laptop computers, tablet personal computers, mainframes, mobile smart phones, smart television, programmable hand-held devices and consumer products, distributed computing infrastructures over a network, cloud computing environments, or any assembly of computing components such as memory and processing—for example.

As illustrated in The Computing Environment FIG, the invention disclosed herein can be implemented over a system that contains a device or unit for processing the instructions of the invention. This processing unit 16000 can be a single core central processing unit (CPU), multiple core CPU, graphics processing unit (GPU), multiplexed or multiply-connected GPU system, or any other homogeneous or heterogeneous distributed network of processors.

In some embodiment of the invention disclosed herein, the computing environment can contain a memory mechanism to store computer-readable media. By way of example and not limitation, this can include removable or non-removable media, volatile or non-volatile media. By way of example and not limitation, removable media can be in the form of flash memory card, USB drives, compact discs (CD), blu-ray discs, digital versatile disc (DVD) or other removable optical storage forms, floppy discs, magnetic tapes, magnetic cassettes, and external hard disc drives. By way of example but not limitation, non-removable media can be in the form of magnetic drives, random access memory (RAM), read-only memory (ROM) and any other memory media fixed to the computer.

As depicted in The Computing Environment FIG, the computing environment can include a system memory 16030 which can be volatile memory such as random access memory (RAM) and may also include non-volatile memory such as read-only memory (ROM). Additionally, there typically is some mass storage device 16040 associated with the computing environment, which can take the form of hard disc drive (HDD), solid state drive, or CD, CD-ROM, blu-ray disc or other optical media storage device. In some other embodiments of the invention the system can be connected to remote data 16240.

The computer readable content stored on the various memory devices can include an operating system, computer codes, and other applications 16050. By way of example not limitation, the operating system can be any number of proprietary software such as Microsoft windows, Android, Macintosh operating system, iphone operating system (iOS), or Linux commercial distributions. It can also be open source software such as Linux versions e.g. Ubuntu. In other embodiments of the invention, data processing software and connection instructions to a sensor device 16060 can also be stored on the memory mechanism. The procedural algorithm set forth in the disclosure herein can be stored on—but not limited to—any of the aforementioned memory mechanisms. In particular, computer readable instructions for training and subsequent image classification tasks can be stored on the memory mechanism.

The computing environment typically includes a system bus 16010 through which the various computing components are connected and communicate with each other. The system bus 16010 can consist of a memory bus, an address bus, and a control bus. Furthermore, it can be implemented via a number of architectures including but not limited to Industry Standard Architecture (ISA) bus, Extended ISA (EISA) bus, Universal Serial Bus (USB), microchannel bus, peripheral component interconnect (PCI) bus, PCI-Express bus, Video Electronics Standard Association (VESA) local bus, Small Computer System Interface (SCSI) bus, and Accelerated Graphics Port (AGP) bus. The bus system can take the form of wired or wireless channels, and all components of the computer can be located remote from each other and connected via the bus system. By way of example and not of limitation, the processing unit 16000, memory 16020, input devices 16120, output devices 16150 can all be connected via the bus system. In the representation depicted in The Computing Environment FIG, by way of example not limitation, the processing unit 16000 can be connected to the main system bus 16010 via a bus route connection 16100; the memory 16020 can be connected via a bus route 16110; the output adapter 16170 can be connected via a bus route 16180; the input adapter 16140 can be connected via a bus route 16190; the network adapter 16260 can be connected via a bus route 16200; the remote data store 16240 can be connected vis a bus route 16230; and the cloud infrastructure can be connected to the main system bus vis a bus route 16220.

In some embodiment of the invention disclosed herein, The Computing Environment FIG illustrates that instructions and commands can be input by the user using any number of input devices 16120. The input device 16120 can be connected to an input adapter 16140 via an interface 16130 and/or via coupling to a tributary of the bus system 16010. Examples of input devices 16120 include but are by no means limited to keyboards, mouse devices, stylus pens, touchscreen mechanisms and other tactile systems, microphones, joysticks, infrared (IR) remote control systems, optical perception systems, body suits and other motion detectors. In addition to the bus system 16010, examples of interfaces through which the input device 16120 can be connected include but are by no means limited to USB ports, IR interface, IEEE 802.15.1 short wavelength UHF radio wave system (bluetooth), parallel ports, game ports, and IEEE 1394 serial ports such as FireWire, i.LINK, and Lynx.

In some embodiment of the invention disclosed herein, The Computing Environment FIG illustrates that output data, instructions, and other media can be output via any number of output devices 16150. The output device 16150 can be connected to an output adapter 16170 via an interface 16160 and/or via coupling to a tributary of the bus system 16010. Examples of output devices 16150 include but are by no means limited to computer monitors, printers, speakers, vibration systems, and direct write of computer-readable instructions to memory devices and mechanisms. Such memory devices and mechanisms can include by way of example and not limitation, removable or non-removable media, volatile or non-volatile media. By way of example and not limitation, removable media can be in the form of flash memory card, USB drives, compact discs (CD), blu-ray discs, digital versatile disc (DVD) or other removable optical storage forms, floppy discs, magnetic tapes, magnetic cassettes, and external hard disc drives. By way of example but not limitation, non-removable media can be in the form of magnetic drives, random access memory (RAM), read-only memory (ROM) and any other memory media fixed to the computer. In addition to the bus system 16010, examples of interfaces through which the output device 16150 can be connected include but are by no means limited to USB ports, IR interface, IEEE 802.15.1 short wavelength UHF radio wave system (bluetooth), parallel ports, game ports, and IEEE 1394 serial ports such as FireWire, i.LINK, and Lynx.

In some embodiment of the invention disclosed herein some of the computing components can be located remotely and connected to via a wired or wireless network. By way of example and not limitation, The Computing Environment FIG shows a cloud 16210 and a remote data source 16240 connected to the main system bus 16010 via bus routes 16220 and 16230 respectively. The cloud computing infrastructure 16210 can itself contain any number of computing components or a complete computing environment in the form of a virtual machine (VM). The remote data source 16240 can be connected via a network to any number of external sources such as NMR spectrometry devices, X-ray diffraction devices, electron microscopes, imaging devices, imaging systems, or imaging software.

In some embodiment of the invention disclosed herein, a sensor system 16060 which captures and pre-processes data is attached directly to the system. For example, this may be an electron microscope (and associated image processing software); it may be a camera in the case of an imaging system, say for processing distance map photographs; or it may be an X-ray crystallography machine or an NMR spectrometer (and associated software), excetera. Stored in the memory mechanism—16020, 16240, or 16210—are machine learning models, algorithms, and data products developed according to the procedures set-forth herein. Computer-readable instructions are also stored in the memory mechanism, so that upon command, protein structure representation data, its substrates and associated data can be captured or can be received over a network from a remote or local previously collated database. This transmission of data can be done over a wired or wireless network as previously detailed, as the source and/or recipient of the data output can be at a remote location.

The objects set forth in the preceding are presented in an illustrative manner for reason of efficiency. It is hereby noted that the above disclosed methods and systems can be implemented in manners such that modifications are made to the particular illustration presented above, while yet the spirit and scope of the invention is retained. The interpretation of the above disclosure is to contain such modifications, and is not to be limited to the particular illustrative examples and associated drawings set-forth herein.

Furthermore, by intention, the following claims encompass all of the general and specific attributes of the invention described herein; and encompass all possible expressions of the scope of the invention, which can be interpreted—as pertaining to language—as falling between the aforementioned general and specific ends.

1. A method, comprising:
a. receiving, at a processor, a neural network trained to classify representations of protein structural conformations,
i. wherein the neural network is equipped with a discriminative feature localization mechanism;
ii. wherein the neural network's output includes a scoring of whether or not the protein structural conformation representation has a given physical or biological property of interest,
iii. wherein the neural network's output includes a discriminative feature localization map;
b. receiving, at a processor, a set of initial values of a plurality of structure parameters specifying the protein's conformational structure;
c. using, via the processor, the trained neural network to perform inference on the initial values of the protein's conformational structure representation,
i. wherein the neural network outputs both the property classification and the discriminative feature map;
d. receiving, at a processor, a local structure update method, which is a set of instructions to update the values of the localized subset of structure parameters specified by the discriminative feature map,
i. wherein the local structure update method consists of a plurality of iterative steps, and some termination criteria,
ii. wherein the output of each iterative update step—an updated conformational structure representation—is evaluated by the neural network, yielding an updated classification score and an updated discriminative feature map,
iii. wherein:
1. if termination criteria are not yet met, then the updated conformational structure representation and the updated discriminative feature map are both re-entered as input into the local update method, else
2. if termination criteria are met, then the local structure update iteration terminates, and the updated conformational structure representation and the updated discriminative feature map are both returned as output.

2. The method of claim 1, wherein the steps of obtaining the neural network comprise:
a. preparing or accessing a dataset consisting of a plurality of protein structure representations, wherein the label of each represented protein conformation specifies whether or not the desired physical or biological property is present (or absent),
i. wherein, the structural conformation of each protein in the dataset is specified via a plurality of structure parameters,
ii. wherein, the dataset consists of a plurality of proteins and a plurality of possible property values across the dataset;
b. configuring the neural network architecture with a discriminative feature localization mechanism, and;
c. using the dataset to train the neural network to classify structural conformations of proteins as either having or lacking the specified desired physical or biological property.

3. The method of claim 1, wherein the termination criteria include: (a) a property determining neural network classification score criterion; and or (b) an iteration count stopping criterion.

4. The method of claim 1, wherein at each iterative step of the localized structure update method, none of the plurality of structure parameters outside of the discriminative feature map are changed.

5. The method of claim 1:
a. wherein the property of interest is a desired property, the method further comprising:
i. in terms of the neural network output scores, the localized structure update method proceeds by iteratively updating the protein structure representation in a manner that moves the classification score towards (or further towards) scores representing the desired property;
b. wherein the property of interest is an undesired property, the method further comprising:
i. in terms of the neural network output scores, the localized structure update method proceeds by iteratively updating the protein structure representation in a manner that moves the classification score away from (or further away from) scores representing the undesired property.

6. The method of claim 1, wherein the objective is to increase the accuracy of a protein structure prediction, wherein the method further comprises:
a. using the protein structure prediction as initial values of the plurality of structure parameters specifying the protein conformational structure;
b. obtaining a neural network trained to classify protein structures as having or lacking some physical or biological property which the protein is experimentally known to have;
c. using that neural network as the scoring neural network in the iterative local structure update procedure;
d. outputting the updated conformational structure of the protein when termination criteria are met.

7. The method of claim 1, wherein there is a plurality of property determining classifier neural networks, one or a plurality per each physical or biological property of interest.

8. The method of claim 1, wherein the local structure update method is constrained by a function to keep the values of the plurality of structure parameters within the bounds of physical feasibility.

9. The method of claim 1, wherein the plurality of structural parameters are a plurality of torsion angles between the amino acids in the amino acid sequence constituting the protein.

10. The method of claim 1, wherein the plurality of structure parameters are atomic coordinates of the amino acids in the amino acid sequence constituting the protein.

11. The method of claim 1, wherein for each of the plurality of structure parameters, the values are specified as a probability distribution over possible values of that structure parameter.

12. The method of claim 1, wherein the discriminative feature localization method is a class activation map (CAM) variant, defined as any method that uses a decomposition of the neural network's class activation pipeline to determine the discriminative feature map; wherein the class activation pipeline comprises the sequence of feature extraction, weighted scalings, and output activation.

13. The method of claim 1, wherein the discriminative feature localization method is occlusion sensitivity analysis.

14. The method of claim 1, wherein the property being assessed by the neural network is whether the protein structure was experimentally determined.

15. The method of claim 1, wherein the local structure update method is particle swarm.

16. The method of claim 1, wherein the local structure update method is a genetic algorithm.

17. A method of detecting a proteinopathy, the method comprising:
a. using the method of claim 1, to determine a final structure of that protein;
b. comparing the predicted structure to the experimentally determined structure of that protein taken from a sample in a human, animal, or plant.

18. The method of claim 1, wherein the objective is to discover and synthesize a ligand drug or a ligand industrial enzyme, the method further comprising:
a. identifying a target protein of interest for the ligand, and obtaining initial values of the predicted structure of that target protein;
b. obtaining a desired property for the target protein;
c. obtaining a final predicted conformational structure of the target protein, by using a predicted structure of the target protein as initial values, and by using the desired property as the property of interest;
d. using the final predicted conformational structure of the target protein to conduct a candidate ligand search or a candidate ligand generation,
i. wherein the candidate ligand search involves assessing the interaction and efficacy of candidate ligands with respect to the predicted structural conformation of the target protein-;
e. synthesizing the ligand.

19. The method of claim 1, wherein the objective is to discover and synthesize a polypeptide ligand for a target protein, the method further comprising:
a. obtaining a representation of the target protein conformational structure;
b. selecting a plurality of candidate polypeptide ligands and their respective conformational structure representations;
c. for each candidate polypeptide ligand, using the efficacy of its interaction with the target protein as the desired property;
d. for each candidate polypeptide ligand, using a predicted conformational structure representation as input initial conditions;
e. for each candidate polypeptide ligand, obtaining a final predicted conformational structure and an associated interaction efficacy;
f. selecting the most efficacious polypeptide ligand from the plurality of candidate polypeptide ligands-;
g. synthesizing the ligand.

20. An apparatus, comprising: a processor and an associated memory, wherein the memory stores instructions that when executed by the processor, cause the processor to:
a. receive a neural network trained to classify representations of protein structural conformations,
i. wherein the neural network is equipped with a discriminative feature localization mechanism;
ii. wherein the neural network's output includes a scoring of whether or not the protein structural conformation representation has a given physical or biological property of interest,
iii. wherein the neural network's output includes a discriminative feature localization map;
b. receive a set of initial values of a plurality of structure parameters specifying the protein's conformational structure;
c. use the trained neural network to perform inference on the initial values of the protein's conformational structure representation,
i. wherein the neural network outputs both the property classification and the discriminative feature map;
d. receive a local structure update method, which is a set of instructions to update the values of the localized subset of structure parameters specified by the discriminative feature map,
i. wherein the local structure update method consists of a plurality of iterative steps, and some termination criteria,
ii. wherein the output of each iterative update step—an updated conformational structure representation—is evaluated by the neural network, yielding an updated classification score and an updated discriminative feature map,
iii. wherein:
1. if termination criteria are not yet met, then the updated conformational structure representation and the updated discriminative feature map are both re-entered as input into the local update method, else
2. if termination criteria are met, then the local structure update iteration terminates, and the updated conformational structure representation and the updated discriminative feature map are both returned as output.
